(19) United States
(12) Patent Application Publication
(10) Pub. No.: US 2005/0244377 A1
(43) Pub. Date: Nov. 3, 2005

(54) Methods of treating cardiac disorders by suppressing the expression of the potassium inwardly-rectifying channel

(76) Inventors: Daniel Sigg, St. Paul, MN (US); James A. Coles JR., Minneapolis, MN (US); Michael Ujhelyi, Maple Grove, MN (US); Rudy Padua, Richfield, MN (US); Daisy P. Cross, Minneapolis, MN (US); William F. Kaemmerer, Edina, MN (US)

Correspondence Address: CAROL M. NIELSEN;WINSTEAD SECHREST & MINICK, P.C., 2400 BANK ONE CENTER, 910 TRAVIS STREET, HOUSTON, TX, 77002, US

(21) Appl. No.: 11/022,368

(22) Filed: Dec. 22, 2004

Related U.S. Application Data
(60) Provisional application No. 60/532,389, filed on Dec. 24, 2003.

(51) Int. Cl. A61K 48/00; C12N 15/86

(52) U.S. Cl. 424/93.2; 514/44; 435/456; 435/325; 536/23.2

(57) ABSTRACT

The present invention is directed toward methods for regulating biological pacemaking activity and devices used in such regulation. Such regulation can be accomplished by introducing genetic material to the heart by transfecting heart cells of the atrium or ventricle with an oligonucleotide, small interfering RNA, that silence KCNJ2, and suppress the I_{K1} current. Suppression (or silencing) of KCNJ2 subsequently induces pacemaker-like activities in previously regular myocytes. This invention provides for methods of targeted delivery using a fluid delivery catheter. Such a catheter allows the targeting of a specific area in the atrium or the ventricle of the heart. Also, combination methods of treating arrhythmia with traditional device-based therapies (e.g., pacemakers and defibrillators) and an oligonucleotide of the subject invention.

### CROSS-REFERENCES TO RELATED APPLICATIONS

The present application claims priority under Title 35, United States Code, § 119 to provisional application U.S. Pat. App. Ser. No. 60/532,389 filed Dec. 24, 2003.

### FIELD OF INVENTION

The present invention relates generally to methods of using oligonucleotides for treating cardiac disease by providing pacemaker function, and more specifically to novel oligonucleotides, constructs and transfected cell lines used to suppress the expression of the potassium inwardly-rectifying channel, subfamily J, member 2 and useful in treating cardiac disease.

### BACKGROUND OF THE INVENTION

The mammalian heart maintains an intrinsic rhythm by creating electric stimuli. Generally, the stimuli form a depolarization wave that propagates within specialized cardiac conducting tissue and the myocardium. The usually well-ordered wave movement facilitates coordinated contractions of the myocardium. These contractions are the engine that moves blood throughout the body. See, generally, The Heart and Cardiovascular System. Scientific Foundations (1986) Fozzard, H. A. et al. eds, Raven Press, NY.

Cardiac electrophysiology has been the subject of intense interest. Generally, the cellular basis for all cardiac electrical activity is the action potential (AP). The AP is conventionally divided into five phases in which each phase is defined by the cellular membrane potential and the activity of potassium, chloride, sodium and calcium ion channel proteins that affect that potential. Propagation of the AP throughout the heart is known to involve gap junctions. See e.g., Tomaselli et al., Cardiovasc. Res. (1999) 42:270 and references cited therein.

Under most circumstances, cardiac stimuli are controlled by recognized physiological mechanisms. Cardiac myogenic tissue is directly effected by both the electrical pulse and the contraction of the tissue. For example, cardiac myocytes exhibit transient increases in cytoplasmic calcium that serve as the driving force behind contraction. Studies have also shown that electrical stimulation of quiescent neonatal cardiac myocytes causes hypertrophy in vitro and activates hypertrophy-associated pathways and certain marker genes. See e.g., McDonough et al., J. Biol. Chem. (1997) 272:24046-14053; McDonough et al., J. Biol. Chem. (1992) 267:11665-11668; Xia et al., J. Biol. Chem. (1998) 273:12593-12598; Xia et al., Proc. Natl. Acad. Sci. (1997) 94:11399-11404; Xia et al., J. Biol. Chem. (2000) 275:1855-1863. Likewise, the ANP and adenylosuccinate synthetase 1 (Adssl) genes are strongly induced by electrical pacing, and their respective promoters can be activated by 10-fold or more by electrical stimulation of myocytes. See e.g., Xia et al., J. Biol. Chem. (1998) 273:12593-12598; Xia et al., J. Biol. Chem. (2000) 275:1855-1863.

In the upper part of the right atrium of the heart is a specialized bundle of neurons known as the sinoatrial node (SA node). Acting as the heart's natural pacemaker the SA node “fires” at regular intervals to cause the heart beat with a rhythm of about 60 to 70 beats per minute for a healthy, resting heart. The electrical impulse from the SA node triggers a sequence of electrical events in the heart to control the orderly sequence of muscle contractions that pump the blood out of the heart. There has been a long-standing recognition that abnormalities of excitable cardiac tissue can lead to abnormalities of the heart rhythm. These abnormalities are generally referred to as arrhythmias. Most arrhythmias are believed to stem from defects in cardiac impulse generation or propagation that can substantially compromise homeostasis, leading to substantial patient discomfort or even death. For example, cardiac arrhythmias that cause the heart to beat too slowly are known as bradycardia, or bradyarrhythmia. In contrast, arrhythmias that cause the heart to beat too fast are referred to as tachycardia, or tachyarrhythmia. See, generally, Cardiovascular Arrhythmias (1973) Dreifus, L. S. and Likoff, W. eds, Grune & Stratton, NY.

The significance of heart related disorders cannot be exaggerated. In the United States alone, cardiac arrest accounts for 220,000 deaths per year. This is thought to account for more than 10% of total American deaths.

Symptoms related to arrhythmias range from nuisance, extra heart beats, to life-threatening loss of consciousness and death. Complete circulatory collapse has also been reported. Morbidity and mortality from such problems continues to be substantial. Atrial fibrillation, a specific form of cardiac arrhythmia, impacts more than 2 million people in the United States. Other arrhythmias account for thousands of emergency room visits and hospital admissions each year. See, e.g., Bosch et al., Cardiovas Res. (1999) 44:121 and references cited therein.

Attempts to treat cardiac arrhythmias and related heart disorders have been largely confined to pharmacotherapy, radiofrequency ablation, use of implantable devices, and related approaches. In particular, radiofrequency ablation has been reported to address a limited number of arrhythmias, e.g., atrioventricular (AV) node re-entry tachycardia, accessory pathway-mediated tachycardia, and atrial flutter. Device-based therapies (pacemakers and defibrillators, for instance) have been reported to be helpful for some patients with bradyarrhythmias and lifesaving for patients with tachyarrhythmias.

There are drugs that regulate arrhythmic events. However, often the effects of a drug are poorly tolerated. Moreover, certain drugs are known to those skilled in the art to carry the risk of mortality. See e.g., Bigger et al., The Pharmacological Basis of Therapeutics 8^{th} supl. ed. (Gilman et al., Eds) McGraw-Hill, NY (1993) and references cited therein.

One alternative to the above-mentioned treatments is gene therapy. Gene therapy holds tremendous promise for treating a wide range of diseases and disorders. In recent years, the ability to control the expression of a delivered therapeutic gene has received much attention. However, unregulated gene expression of a delivered gene has been shown to potentially have detrimental effects in target tissues.

Studies have shown that electrical pacing can regulate gene expression of myogenic cells in vitro and in vivo, probably by activating hypertrophy-associated pathways. See, McDonough et al., J. Biol. Chem. (1997) 272:24046-14053; Xia et al., J. Biol. Chem. (1998) 273:12593-12598; Xia et al., Proc. Natl. Acad. Sci. (1997) 94:11399-11404.

The hypertrophy-associated signal pathways are, however, quite complex and not fully described. Pacing of myogenic cardiac tissue may have at least two components including a direct effect of the electrical pulse and secondary effect of enhanced contraction or rate of contraction; the latter may involve calcium transients. Previous studies have also shown that electrical stimulation of quiescent neonatal cardiac myocytes causes hypertrophy in vitro and activates marker genes. McDonough et al., J. Biol. Chem. (1997) 272:24046-14053. The ANP and adenylosuccinate synthetase 1 (Adssl) genes are strongly induced by pacing, and their respective promoters can be activated by a 10-fold or more by electrical stimulation of myocytes See e.g., Xia et al., J. Biol. Chem. (1998) 273:12593-12598; Xia et al., J. Biol. Chem. (2000) 275:1855-1863.

Recent experimental results have also shown that genetically-engineered pacemakers could be developed as a possible alternative to implantable devices. See, Miake et al., Nature (2002) 419:132 and Rosen et al., Circulation (2003) 107(8):1106-1109.

A need exists therefore for new methods of treating cardiac pacing dysfunction and arrhythmic events via expression and suppression of genes.

### SUMMARY OF THE INVENTION

The present invention is directed toward methods of regulating biological pacemaking activity through suppression of potassium inwardly-recifying channel, subfamily J, member 2 gene (“KCNJ2”) gene locus 17q23.1-q24.2. The protein encoded by this gene (potassium inwardly-rectifying channel KIR2) has a greater tendency to allow potassium to flow into a cell rather than out of a cell and regulates the I_{K1} current. Suppression of KCNJ2 provides alone or in combination with other biological agents and/or devices about 70 to 100 percent block of I_{K1} current. Furthermore by blocking pacemaker KCNJ2 expression, pacemaker activity can be induced in regions within the heart that do not normally provide such pacemaker function when KCNJ2 is expressed in those regions.

Regulation of pacemaker activity in the heart may be accomplished with small interfering RNA (“siRNA), otherwise referred to herein as an oligonucleotide, silencing KCNJ2.

This invention provides methods of targeted delivery using a fluid delivery catheter. Such a catheter allows the targeting of a specific area in the atrium or the ventricle of the heart. The fluid delivery catheter may be used to deliver the oligonucleotide, constructs and/or host cells to particular regions of the heart.

This invention provides for methods of treating arrhythmias comprising administering one or more oligonucleotides in combination with traditional device-based therapies (e.g., pacemakers and defibrillators) and/or other biologically active agents.

### BRIEF DESCRIPTION OF DRAWINGS

For a more complete understanding of the present invention, and the advantages thereof, reference is now made to the following descriptions taken in conjunction with the accompanying drawings, in which:

FIG. 1 is a diagram of a human heart.

FIG. 2 illustrates in silico data demonstrating automaticity of ventricular cells using a Luo-Rudy model if I_{K1} current is blocked;

FIG. 3 illustrates post-transcriptional gene silencing initiated by siRNA;

FIG. 4 illustrates a method of using adeno-associated viral vectors to deliver siRNA into the vascular system;

FIGS. 5 A and B illustrate pharmacologically-responsive (A) and electrically responsive (B) plasmid constructs for regulating expression with siRNA;

FIG. 6 is a schematic diagram of a right side of a heart, similar to FIG. 1, in which a guiding catheter is positioned for delivery of the genetic construct of the invention;

FIG. 7 illustrates a representative viral expression cassette comprising siRNA which can serve to suppress KCNJ2 according to some embodiments of the present invention;

FIG. 8 illustrates a representative adeno-associated viral expression cassette comprising siRNA which can serve to suppress KCNJ2 according to some embodiments of the present invention;

FIG. 9 illustrates a eGFPviral control vector used for canine gene expression experiments; and

FIG. 10 is a fluorescence microscope image demonstrating positive GFP expression 4 weeks after injection of rAAV2-eGFP into canine myocardium (width of image =18 mm).

### DETAILED DESCRIPTION OF THE INVENTION

Regulation of pacemaker activity in the heart may be accomplished with small interfering RNA (“siRNA), otherwise referred to herein as an oligonucleotide, silencing KCNJ2. The protein encoded by KCNJ2, potassium inwardly-rectifying channel KIR2, regulates the I_{K1} current.

RNA interference is a post-transcriptional process triggered by the introduction of double-stranded RNA which leads to gene silencing in a sequence-specific manner. RNA interference reportedly occurs naturally in organisms as diverse as nematodes, trypanosmes, plants and fungi. It is believed to protect organisms from viruses, modulate transposon activity and eliminate aberrant transcription products. RNA interference is an important method for analyzing gene function in eukaryotes and as used in the subject invention has been developed as a therapeutic method of gene silencing for heart disease.

Small interfering RNA or siRNA, typically 19-23 base pair, double-stranded RNA of synthetic origin are designed to be specific for the nucleotide sequence of its intended target in mRNA. The siRNA interacts with a protein complex that has a helicase activity and a nuclease activity, known as “RNA-induced silencing complex (RISC).” RISC unwinds the double-stranded siRNA via its helicase activity. The resulting single-stranded RNA binds to the RISC complex and the RNA guides the complex to the target RNA with the complementary sequence. The target RNA is then cleaved by the nuclease activity of RISC. The target RNA is further degraded by cellular nucleases. This process is known as RNA interference or RNAi.

While the terms used herein are well known to those of skill in the art, the following definitions are provided to facilitate an understanding of the invention:

The term “polynucleotide” refers to a molecule having a sequence of two or more nucleotides such as an oligonucleotide and fragments or portions thereof, as well as peptide nucleic acids (PNA), fragments, portions or antisense molecules thereof, and to DNA or RNA of genomic or synthetic origin which can be single- or double-stranded, and represent the sense or antisense strand.

DNA or deoxyribonucleic acid has deoxyribose as the sugar group and nitrogenous bases: adenine (A), guanine (G), thymine (T), and cytosine (C).

RNA or ribonucleic acid has ribose as the sugar group, and the nucleotide bases: adenine (A), guanine (G), uracil (U), and cytosine (C).

The term “messenger RNA” or “mRNA” refers to RNA that serves as a template for protein synthesis.

The term “gene” refers to a hereditary unit, a sequence of DNA, occupying a specific location on a chromosome and may be a segment of DNA that is involved in producing a polypeptide chain and can include regions preceding and following the coding DNA as well as introns and exons.

The term “gene expression refers to conversion of information encoded in a gene first into messenger RNA (“mRNA”) and then to a protein. As used herein, expressed genes may include DNA transcribed into mRNA, but not translated into protein (e.g., transfer and ribosomal RNAs). Gene suppression or silencing is an attenuation of gene expression.

The term “coding sequence” refers to a sequence of DNA that is transcribed into RNA or a sequence of mRNA translated into a polypeptide, in vitro or in vivo.

The term “ion channel protein” refers to a protein that transports ions across cell membranes.

The term “plasmid” refers to a circular DNA molecule typically found in bacteria.

The term “cDNA” refers to nucleic acids that share the arrangement of sequence elements found in native mature mRNA species, where sequence elements are exons (sequences encoding open reading frames of the encoded polypeptide) and 3′ and 5′ non-coding regions. Normally mRNA species have contiguous exons, with the intervening introns removed by nuclear RNA splicing, to create a continuous open reading frame encoding the polypeptide of interest.

The term “construct” is used interchangeably herein with the terms “vector” and “plasmid” and refers to a DNA molecule that is used to deliver a specific gene(s) into a target cell.

The term “promoter” refers to a sequence of nucleotides that direct transcription in a cell. “Promoter” is also meant to encompass those elements sufficient for promoter-dependent gene expression controllable for cell-type specific expression, tissue-specific expression or inducible by external signals or agents.

The term “enhancer” refers to DNA sequences that increase transcription of genes. Enhancers usually function in either 3′ to 5′ or 5′ to 3′ orientation and at various distances from a promoter.

The term “PCR,” or “polymerase chain reaction,” refers to a system for in vitro amplification of DNA wherein two synthetic oligonucleotide primers, which are complementary to two regions of the target DNA (one for each strand) to be amplified, are added to the target DNA in the presence of excess deoxynucleotides and Taq polymerase, a heat stable DNA polymerase. In a series of temperature cycles, the DNA is repeatedly denatured, annealed to the primers, and a daughter strand extended from the primers. As the daughter strands act as templates in subsequent cycles, amplification occurs in an exponential fashion.

The term “dominant-negative” refers analogously to what is sometimes referred to as a “poison pill” which can be driven (i.e., expressed) by an appropriate DNA construct to produce a dominant negative protein having the capacity to inactivate an endogenous protein.

The term “gap junction” refers to small pore-like structures that connect cardiac muscle cells to each other.

The term “transformation” refers to a permanent or transient genetic change, typically a permanent genetic change, induced in a cell following incorporation of new nucleic acid (e.g., DNA or RNA exogenous to the cell). Genetic change can be accomplished either by incorporation of the new nucleic acid into the genome of the host cell, or by transient or stable maintenance of the new DNA as an episomal element.

The terms “transformed cell,” “transfected cell,” “transduced cell,” “recombinant cell” or “host cell” refer to a cell or an ancestor of a cell into which a DNA molecule has been introduced or incorporated by means of recombinant DNA techniques.

A “vector” or “expression vector” is a DNA molecule that is used to carry a specific gene into a target cell. Once the expression vector is inside the cell, the protein that is coded for by the gene is produced by normal transcription and translation process of the host cell. Expression vectors are often specifically designed to contain non-protein coding sequences that act as enhancers and promoter regions and allow for efficient transcription of the gene that is carried on the expression vector.

In gene therapy, a “vector” is vehicle for delivery of genetic material such as DNA to a cell. For example, a virus itself may serve as a vector, if it has been re-engineered and is often used to deliver a gene to its target cell.

As applied to genetics more generally, a vector is a construct such as a plasmid or a bacterial artificial chromosome that contains an origin of replication. DNA by itself may be regarded as a vector or construct when it is used for cell transformation. Appropriate replication causes a cell to copy the construct along with the cell's chromosomes and to pass it along to its progeny.

“MV” is an adeno-associated virus vector. Adeno-associated virus is not known to cause any disease in humans, and upon transducing cells in vitro or in vivo, it produces long-lasting expression in the cells of genes on the DNA delivered by the MV vector.

“AV” is an adenovirus vector.

“Lentivirus” is a virus, such as HIV, that incorporates its passenger genes into non-dividing cells.

“Liposome” refers to a synthetic lipid bilayer vesicle that fuses with a cell's outer membrane and is used to transport molecules into cells.

The term “ribozyme” refers to RNA molecules which can act as enzymes; that is, possess catalytic activity and can specifically cleave (cut) other RNA molecules.

The term “antisense” refers to a strand of DNA molecule whose sequence is complementary to the strand represented in mRNA. The term also refers to an RNA molecule complementary to the strand normally processed into mRNA and translated to a protein. Antisense RNA hybridizes to mRNA, physically blocks mRNA translation and targets the mRNA for destruction by cellular nucleases.

The term “electrical coupling” refers to the interaction between cells which allow for intracellular communication between cells so as to provide for electrical conduction between the cells. Electrical coupling in vivo provides the basis for, and is generally accompanied by, electromechanical coupling, in which electrical excitation of cells through gap junctions in the muscle leads to muscle contraction.

The term “dicer” refers to an endonuclease enzyme capable of cleaving long, double-stranded RNA molecules into fragments of 21-23 base pairs.

In a normal human heart, for example, cardiac contraction is initiated by the spontaneous excitation of the sinoatrial (SA) node that is located in the right atrium. The electrical current generated by the SA node travels to the atrioventricular (AV) node where it is then transmitted to the bundle of His and Purkinje network, which branches in many directions to facilitate simultaneous contraction of the left and right ventricles.

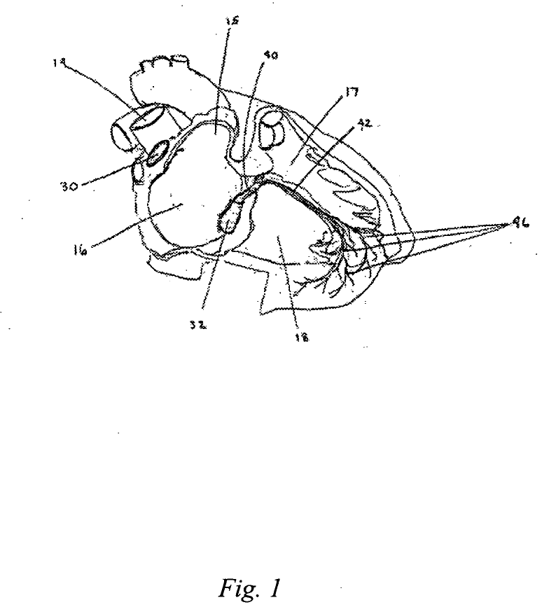

FIG. 1 is a schematic diagram of a right side of a heart having an anterior-lateral wall peeled back to expose a portion of a heart's intrinsic conduction system and chambers of a right atrium (RA) 16 and a right ventricle (RV) 18. Pertinent elements of the heart's intrinsic conduction system, illustrated, in FIG. 1, include a sinoatrial (SA) node 30, an atrioventricular (AV) node 32, a bundle of His 40, a right bundle branch 42, left bundle branches (not shown) and Purkinje fibers 46. SA node 30 is shown at a junction between a superior vena cava 14 and RA 16. An electrical impulse initiated at SA node 30 travels rapidly through RA 16 and a left atrium (not shown) to AV node 32. At AV node 32, the impulse slows to create a delay before passing on through a bundle of His 40, which branches, in an interventricular septum 17, into a right bundle branch 42 and a left bundle branch (not shown) and then, apically, into Purkinje fibers 46. Following the delay, the impulse travels rapidly throughout RV 18 and a left ventricle (not shown). Flow of the electrical impulse described herein creates an orderly sequence of atrial and ventricular contraction to efficiently pump blood through the heart. When a portion of the heart's intrinsic conduction system becomes dysfunctional, efficient pumping is compromised.

Typically, a patient whose SA node 30 has become dysfunctional, may have an implantable pacemaker system implanted wherein lead electrodes are placed in an atrial appendage 15. The lead electrodes stimulate RA 16 downstream of dysfunctional SA node 30 and the stimulating pulse travels on to AV node 32, bundle of His 40, and Purkinje fibers 46 to restore physiological contraction of the heart. However, if a patient has a dysfunctional AV node 32, pacing in atrial appendage 15 will not be effective, since it is upstream of a block caused by the damage.

Pacing at the bundle of His 40 provides the advantage of utilizing the normal conduction system of the heart to carry out ventricular depolarizations. In other words, stimulation provided at the bundle of His will propagate rapidly to the entire heart via the right bundle 42, the left bundle (not shown), and the Purkinje fibers. This provides synchronized and efficient ventricular contraction, unlike pacing from the apex of the right ventricle where the electrical activity propagates at a slower rate because myocardial tissue is a slow conductor compared to the rapidly conducting Purkinje network.

Like cells of other excitable tissue in the body, cardiac cells allow a controlled flow of ions across the membranes. This ion movement across the cell membrane results in changes in transmembrane potential (depolarization), which is a trigger for cell contraction. The heart cells are categorized into several cell types (e.g. atrial, ventricular, etc.) and each cell type has its own characteristic variation in membrane potential. For example, ventricular cells have a resting potential of ˜−85 mV. In response to an incoming depolarization wave front, these cells fire an action potential with a peak value of ˜20 mV and then begin to repolarize, which takes ˜350 ms to complete. In contrast, SA nodal cells do not have a stable resting potential and instead begin to spontaneously depolarize when their membrane potential reaches ˜−50 mV. Cells, such as SA nodal cells, that do not have a stable resting transmembrane potential, but instead increase spontaneously to the threshold value, causing regenerative, repetitive phase 4 depolarization, are said to have automaticity.

Cardiac muscle cells are structurally connected to each other via small pore-like structures known as gap junctions, so that when a few cardiac cells depolarize, they act as a current source to adjacent cells causing them to depolarize as well; and these cells in turn relay the electrical charge to adjacent cells. Once depolarization begins within a mass of cardiac cells, it spreads rapidly by cell-to-cell conduction until the entire mass is depolarized causing a mass of cardiac cells to contract as a unit.

The cells in the SA node are specialized pacemaker cells and have the highest firing rate. Depolarization from these cells spreads across the atria. Since atrial muscle cells are not connected intimately with ventricular muscle cells, conduction does not spread directly to the ventricle. Instead, atrial depolarization enters the AV node, and after a brief delay, is passed on to the ventricles via the bundle of His and Purkinje network, initiating cellular depolarization along the endocardiuim. Depolarization then spreads by cell-to-cell conduction throughout the entire ventricular mass.

The SA node's unique cells include a combination of ion channels that endow it with its automaticity. A review of the features of cardiac electrical function and description of the current understanding of the ionic and molecular basis, thereof, can be found in Schram et al., Circulation Research (2002) May 17, pp. 939-950.

Some of the unique features of the SA node cells include the absence of Na⁺ channels and the absence of inwardly rectifying K⁺ (I_{K1}) channels. In the absence of sodium current, the upstroke of SA node action potential is primarily mediated by L-type Ca²⁺ channels (I_{CaL}). SA node cells do not have a stable resting potential because of its unique distribution of ion channels (e.g., lack of I_{K1}, HCN expression) and begin to depolarize immediately after the repolarization phase is complete. The maximum diastolic potential for SA node cells is approximately −50 mV compared to −78 mV and −85 mV for atrial and ventricular cells, respectively. The slow depolarization phase is mediated by activation of hyperpolarization-activated cyclic nucleotide gated channels (HCN, If current) and T-type Ca²⁺ channels and deactivation of slow and rapid potassium (I_{Ks} and I_{Kr}, respectively), as well as the lack of I_{K1}. The rate of pacemaker discharge in the SA node in a normally functioning heart is approximately in the range of about 60 to 100 beats per minute. In the diseased state, the ability of the SA node to properly pace the heart can be severely compromised.

Regulation of pacemaker function is accomplished by administering the oligonucleotides of the subject invention to a patient, silencing KCNJ2. This gene encodes for the protein known as inward rectifier potassium channel KIR2.1 (Kir2.1, gene locus 17q23.1-q24.2) and also referred to as: potassium inwardly-rectifying channel J2, inward rectifier potassium channel 2; and cardiac inward rectifier potassium channel. The inward potassium rectifier channel 2.1 regulates the I_{K1} current. The oligonucleotides of the subject invention (“siRNA”) suppress the expression of KCNJ2 and therefore greatly reduce the I_{K1} current because the inward rectifier potassium channels KIR2.1 are greatly reduced in number on the cell membrane. The methods of the subject invention also can serve to mimic pacemaker function in other cells in which inward rectifier potassium channel KIR2.1 are typically present.

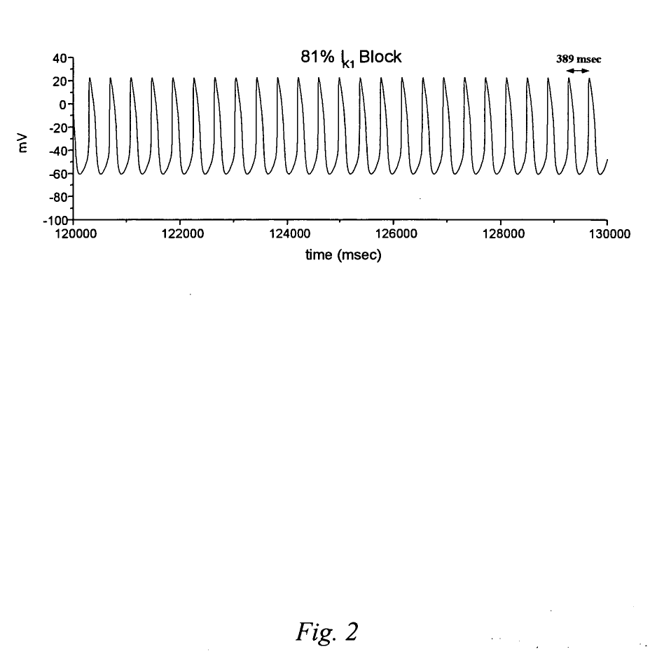

In silico data has shown when I_{K1} current is 80 percent blocked, automatic pacemaker function is created (FIG. 2). Thus, suppression of KCNJ2 encoding for inward rectifier potassium channel KIR2.1 must block 80 percent of I_{K1} current, either alone or in combination with other biological agents and/or devices. Further, in cellular regions within the heart that normally express KCNJ2, a new pacemaker function can be induced in these regions of the heart by suppressing the expression of KCNJ2 in the cells of the region.

RNA interference (RNAi) stops or silences the transcript (mRNA) of an active gene. Small interfering RNA (siRNA) is a double-stranded form of RNA that typically contain between 19 to 23 base pairs (bp), and is specific for the messenger RNA (mRNA) of the target. See, Hannon, Nature (2002) 418:244-51; Sharp, Genes Dev (2001) 15:485-490. Sequence specificity of siRNA is stringent, as single base pair mismatches between the siRNA and its target mRNA dramatically reduce silencing. See Elbashir et al., Nature (2001) 411:494-498; Elbashir et al., EMBO J. (2001) 20: 6877-6888.

In plant and drosophila cells, the generation of siRNAs has been well elucidated. Dicer, a ribonuclease III enzyme, degrades double-stranded RNA to generate smaller RNA molecules, for example microRNA and siRNA. Dicer delivers the siRNA to the RNA-induced silencing complex (RISC). See, Elbashir EMBO J. (2001) 20: 6877-6888; Bernstein et al., Nature (2001) 409:363-366; Hammond et al., Nature Rev Gen (2001) 2: 110-119; Hannon Nature (2002) 418:244-51; see also, Kitabwalla et al., N Engl J Med (2002) 347 (17): 1364-1367. Homology between the antisense portion of the siRNA sequence and the mRNA enables binding of the siRNA to the mRNA. Following this binding, the mRNA is either cleaved or prevented from being translated. Transfection of cells with synthetic siRNA has been shown to elicit highly sequence-specific RNA interference in a sequence-specific manner suggesting that dicer-mediated mechanisms are not essential for loading siRNA molecules into the RISC complex. Elbashir et al., Nature (2001) 411: 494-498.

Therefore, while there are many methods used to design and optimize siRNA, one such method includes the use of a dicer. Here, in the initiation step, the dicer digests double-stranded RNA (dsRNA) into 19-23 nucleotide fragments (siRNAs). The dicer is a member of the RNase III family of dsRNA-specific ribonucleases. The dicer progressively cleaves dsRNA either introduced directly or via a transgene or virus in an ATP-dependent, processive manner. Successive cleavage events degrade the RNA to 19-23 base pair (bp) duplexes (siRNAs), each with 2-nucleotide 3′ overhangs. See, Bernstein et al., Nature (2001) 409:363-366; Hammond et al., Nature Rev Gen (2001) 2: 110-119; Hutvagner et al., Curr Opin Genetics & Development (2002) 12:225-232; Sharp, Genes Dev (2001) 15: 485-490. Whether an siRNA is produced by dicer activity acting on a long double stranded RNA, or an siRNA is introduced into the cell in the form of a short double-standed RNA, the potency of RNA interference is higher than that of antisense oligonucleotides because each RISC complex may cleave and cause the degradation of multiple molecules of the target mRNA. See, Hammond et al., Nature Rev Gen (2001) 2: 110-119; Hutvagner et al., Curr Opin Genetics & Development (2002) 12:225-232; Sharp, Genes Dev (2001) 15: 485-490.

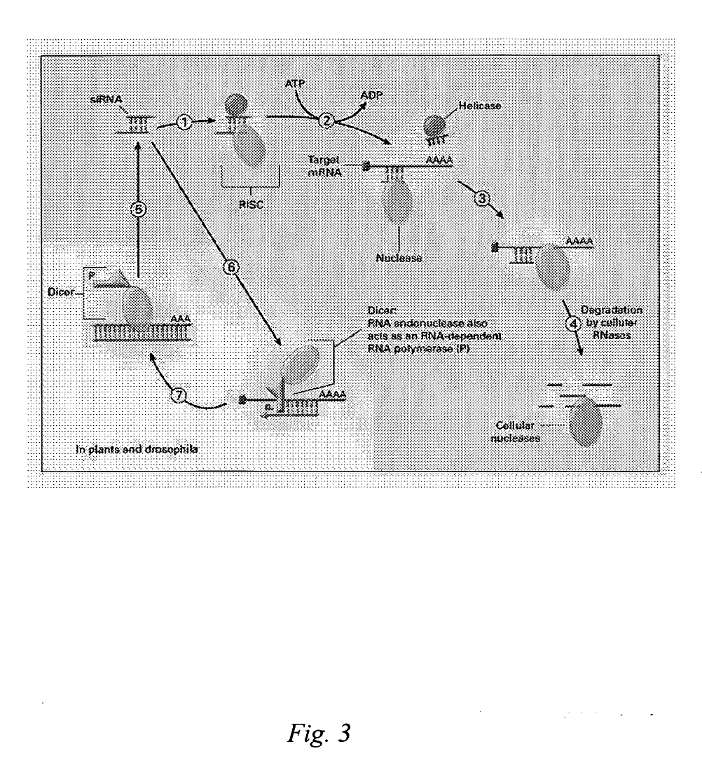

FIG. 3 is illustrative of post-transcriptional gene silencing by siRNA. Small interfering RNA (siRNA) interact with a protein complex with a component with helicase activity and a component with nuclease activity to form a complex termed RNA-induced silencing complex (RISC). Bernstein et al., Nature (2001) 409:363-366. Helicase in RISC uses adenosine triphosphate (ATP) to unwind siRNA, enabling the antisense strand to bind to its target in messenger RNA (mRNA). See, Brown et al., TechNotes (2002) 9(1): 3-5. Nuclease in RISC cleaves the mRNA which is then quickly degraded by other RNases. See, Caplen et al., Proc. Natl. Acad. Sci. USA (2001) 98: 9746-9747; Elbashir et al., Nature (2001) 411: 494-498.

Referring to FIG. 3, in the effector step, the siRNA duplexes bind to helicase and nuclease molecules to form a complex termed “RNA-induced silencing complex,” or RISC. An ATP-dependent unwinding of the siRNA duplex is required for activation of the RISC. The active RISC then targets the homologous transcript by base pairing interactions and cleaves the mRNA at approximately 12 nucleotides from the 3′ terminus of the siRNA. See, Hammond et al., Nature Rev Gen (2001) 2: 110-119; Hutvagner et al., Curr Opin Genetics & Development (2002) 12:225-232; Nykanen et al., Cell (2001) 107:309-321; Sharp, Genes Dev (2001) 15: 485-490. Each RISC has been thought to contain a single siRNA and an RNase that is distinct from dicer. See e.g., Hutvagner et al., Curr Opin Genetics & Development (2002) 12:225-232.

The effectiveness of siRNAs varies—the most potent siRNAs result in greater than 90% reduction in target RNA and protein levels. See e.g., Caplen et al., Proc. Natl. Acad. Sci. USA (2001) 98: 9746-9747; Elbashir et al., Nature (2001) 411: 494-498; Holen et al., Nucleic Acids Research (2002) 30(8):1757-1766. Certain proven siRNAs that have been shown to be very effective contain 21 bp dsRNAs with 2 short 3′ overhangs. See Table 1 below. The effectiveness of the siRNA depends on structure and position. See Brown et al., TechNotes (2002) 9(1): 3-5; Holen et al., Nucleic Acids Research (2002) 30(8):1757-1766; Jarvis et al., TechNotes (2001) 8(5):3-5.

By way of contrast, Table 1 also shows double-stranded RNA longer than 30 bp induces oligoadenylate synthetase and triggers nonspecific RNA degradation and inhibition of protein synthesis. (TABLE 1). TABLE 1 further describes other conventional RNA-targeted interventions in mammalians including small interfering RNA.

TABLE 1
MECHANISMS FOR THE INHIBITION OF RNA IN MAMMALS*

| VARIABLE | SMALL INTERFERING RNA | INTERFERON | ANTISENSE RNA | Ribozyme |
|---|---|---|---|---|
| Inducer of | Double-stranded RNA of | Double-stranded RNA | Antisense single- | RNA of 40-160 bp, a |
| Inhibition | pagozeme 21-23 bp with | of >30 bp | stranded RNA† | specifically |
| | short 3′ overhangs | | | folded |
| | | | | structure that |
| | | | | mimics the |
| | | | | enzymatic |
| | | | | function of |
| | | | | RNase |
| Mechanism | RISC is formed, which | Interferon induces 2′,5′ | Antisense RNA | Ribozymes |
| | consists of small | oligoadenylate | hybridizes with | cleave |
| | interfering RNA, helicase, | synthetase, which | mRNA (sense | specific |
| | and nuclease; requires | activates RNaseL to | RNA); this | phospho- |
| | ATP. | degrade RNA. | physically blocks | diester bonds |
| | RISC unwinds the double- | Interferon and double- | access of mRNA | using base- |
| | stranded small interfering | stranded RNA | (now a double- | pairing and |
| | RNA; the antisense binds | activate protein | stranded RNA) to | their |
| | to target RNA, which is | kinase PKR, which | the translational | interactions to |
| | then cleaved by RISC; | phosphorylates | machinery and | align the |
| | target RNA is further | translation initiation | targets mRNA | ribozyme |
| | degraded by cellular | factor eIF2α, | for destruction by | active site |
| | nucleases. This process is | leading to the | cellular | with the |
| | termed RNA interference. | inhibition of | nucleases. | target site on |
| | | translation. | | the RNA. |
| Specificity | RNA binding and | Neither response is | RNA binding and | Ribozyme |
| | destruction are highly | sequence-specific. | destruction are | binding and |
| | sequence specific. | | sequence- | cleavage are |
| | | | specific. | sequence- |
| | | | | specific. |

The present invention provides oligonucleotides (siRNA) that suppress the of KCNJ2 and block the I_{K1} current. Sequences of the oligonucleotides useful in suppressingKCNJ2 are TABLE 2.

TABLE 2

| | | | | |
|---|---|---|---|---|
| | | | | Estimated |
| | Starting | | | likelihood of |
| | position in | | % G/C | single- |
| Candidate | NM_00891 | Sequence (21 bases) | content | strandedness |

| | | | | |
|---|---|---|---|---|
| 1 | 580 | AACGGTACCTCGCAGACATCT | 52.4 | 61.8 |
| | | (SEQ ID NO: 1) | | |
| 2 | 1421 | AACTTACGAAGTCCCCAACAC | 47.6 | 59.1 |
| | | (SEQ ID NO: 2) | | |
| 3 | 806 | AACCATAGGCTATGGTTTCAG | 42.9 | 34.9 |
| | | (SEQ ID NO: 3) | | |
| 4 | 1515 | AATGAAGTTGCCCTCACAAGC | 47.6 | 29.4 |
| | | (SEQ ID NO: 4) | | |
| 5 | 1134 | AATGTTGGGTTTGACAGTGGA | 42.9 | 25.7 |
| | | (SEQ ID NO: 5) | | |
| 6 | 975 | AATGCCGTGATTGCCATGAGA | 47.6 | 19.5 |
| | | (SEQ ID NO: 6) | | |

The criteria used in selecting these candidate sequences were as follows. that these candidate sequences in fact suppress expression of the target

Below are the candidate DNA sequences that are specified for into a plasmid or MV vector or other gene delivery vector. This genetic of the vector allows the vector to deliver to a cell the DNA that will cause roduce short, interfering RNA that will target the mRNA sequence for Kir2.1. of the interfering RNA in the cell will thereby cause RNA interference g the expression of the Kir2.1 protein (KCNJ2 gene product).

| | | | Reverse complement of the | RNA polymerase III |
|---|---|---|---|---|
| Candidate | SEQ ID NO: | Loop | target sequence | termination signal |
| 1 | 1 | TTCAAGAGA | AGATGTCTGCGAGGTACCGTT | TTTTTT |
| 2 | 2 | TTCAAGAGA | GTGTTGGGGACTTCGTAAGTT | TTTTTT |
| 3 | 3 | TTCAAGAGA | CTGAAACCATAGCCTATGGTT | TTTTTT |
| 4 | 4 | TTCAAGAGA | GCTTGTGAGGGCAACTTCATT | TTTTTT |
| 5 | 5 | TTCAAGAGA | TCCACTGTCAAACCCAACATT | TTTTTT |
| 6 | 6 | TTCAAGAGA | TCTCATGGCAATCACGGCATT | TTTTTT |

The DNA and exact length of the loop sequence in the above examples are only one of many possible loop sequences which serve to physically separate the target sequence from its reverse complement. The reverse complement of the target sequence is the sequence that corresponds to the complement of the DNA bases in the target sequences following Watson-Crick base pairing rules, in the reverse order as the target sequence. Finally, the RNA polymerase III termination signal must be six or more T bases in series, as shown, and further, six or more T bases cannot appear elsewhere in this DNA sequence for producing siRNA unless premature termination of the RNA transcript occur.

The resulting vector DNA sequences that encode for RNA hairpin transcripts corresponding to each of the siRNA candidates targeting Kir2.1 mRNA are the concatenation of the siRNA target sequence, a loop sequence, the reverse complement of the siRNA target sequence, and the RNA pol III termination signal, as follows:

| | |
|---|---|
| Sequence | |
| number | vector DNA sequence |

| | |
|---|---|
| 7 | AACGGTACCTCGCAGACATCTTTCAAGAGAAGATGTCTGCGAGGTACCGTTTTTTTT |
| 8 | AACTTACGAAGTCCCCAACACTTCAAGAGAGTGTTGGGGACTTCGTAAGTTTTTTTT |
| 9 | AACCATAGGCTATGGTTTCAGTTCAAGAGACTGAAACCATAGCCTATGGTTTTTTTT |
| 10 | AATGAAGTTGCCCTCACAAGCTTCAAGAGAGCTTGTGAGGGCAACTTCATTTTTTTT |
| 11 | AATGTTGGGTTTGACAGTGGATTCAAGAGATCCACTGTCAAACCCAACATTTTTTTT |
| 12 | AATGCCGTGATTGCCATGAGATTCAAGAGATCTCATGGCAATCACGGCATTTTTTTT |

Generally, proteins, polynucleotides and oligonucleotides sequences can be obtained from a variety of sources including, but not limited to, GenBank (National Center for Biotechnology Information (NCBI)), EMBL data library, SWISS-PROT (University of Geneva, Switzerland), the PIR-International database; and the American Type Culture Collection (ATCC) (10801 University Boulevard, Manassas, Va. 20110-2209). See generally Benson, D. A. et al. (1997) Nucl. Acids. Res. 25:1 for a description of Genbank.

Suppression of KCNJ2 in vitro and in vivo is a matter for empirical testing.
Sequences are within the coding region of the Kir2.1 mRNA (between bases 384 and 1667, using the numbering as in NM_{—}000891).
Sequences consist of two consecutive A's and the 19 successive bases, for a total of 21 bases.
The G/C content of the targets is between 40% and 60%.
Sequences have at least 4 mismatches with other genes or genomic sequences found elsewhere in the human genome (based on the current data searchable via the BLAST software at the National Center for Biotechnology Information public website, PubMed). This is to avoid potential off-target gene silencing, to the extent predictable.
There are a maximum of three consecutive T's or A's within the target sequences (to avoid possible premature termination of an siRNA hairpin transcript produced by RNA polymerase type III).
Sequences are rank ordered according to the predicted likelihood that the target mRNA molecule is single-stranded in this region of the mRNA, based on the MFOLD software (publicly available on various websites). This is to maximize likely accessibility of the target mRNA region by the siRNA complex, although there is some evidence that this consideration is not necessary (in that the siRNA complex apparently has intrinsic helicase activity).

Thus, the present invention provides for methods by which siRNA can be used to suppress or silence KCNJ2. The various therapeutic uses of compounds, constructs or cells of the subject invention include a variety of cardiac disorders and/or conditions in which suppression of KCNJ2 is desired. Exemplary diseases amenable to treatment by such methods include, but are not limited to, Anderson syndrome, arrhythmias (ventricular tachycardia), congestive heart failure, and heart block.

The present invention also provides for methods of delivery of the oligonucleotides via vectors and constructs to the myocardium and other cells, including, but are not limited to, cardiac phenotype such as adult or embryonic cardiac stem cells, isolated cardiac myocytes, in vivo and in vitro. Oligonucleotides can be injected directly into the myocardium as described by Guzman et al., Circ. Res. (1993) 73:1202-1207. Cells may be transfected or transformed with constructs that contain the oligonucleotides of the subject invention in vivo or ex vivo.

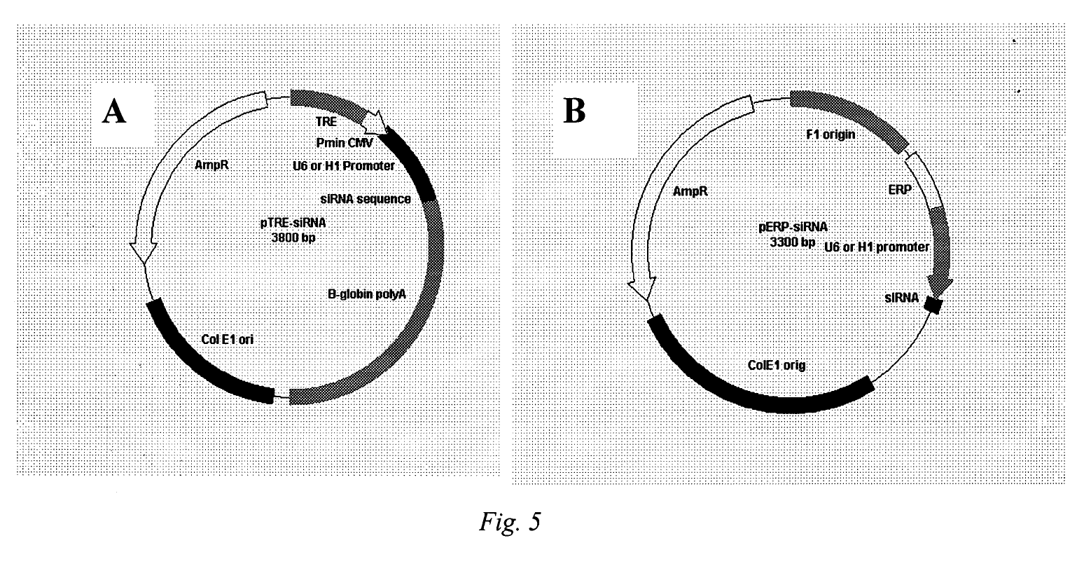

FIG. 5 depicts two suitable constructs useful in connection with the methods of the subject invention. One example plasmid (FIG. 5A) is a pharmacologically-controlled system which expresses the siRNA under the control of an siRNA promoter fused to a tet-responsive minimal CMV (P_{hCMv*-1}) promoter. The tetracycline responsive element (TRE) promoter activity is enhanced by the binding of constitutively-produced rtTA or tTA regulatory proteins (expressed on a separate plasmid construct, not shown), and by exogenously adding doxycycline. In FIG. 5B, an electrically-responsive promoter element is fused upstream of the siRNA promoter and siRNA sequence. Here, electrical stimulation activates the promoter to enhance siRNA expression.

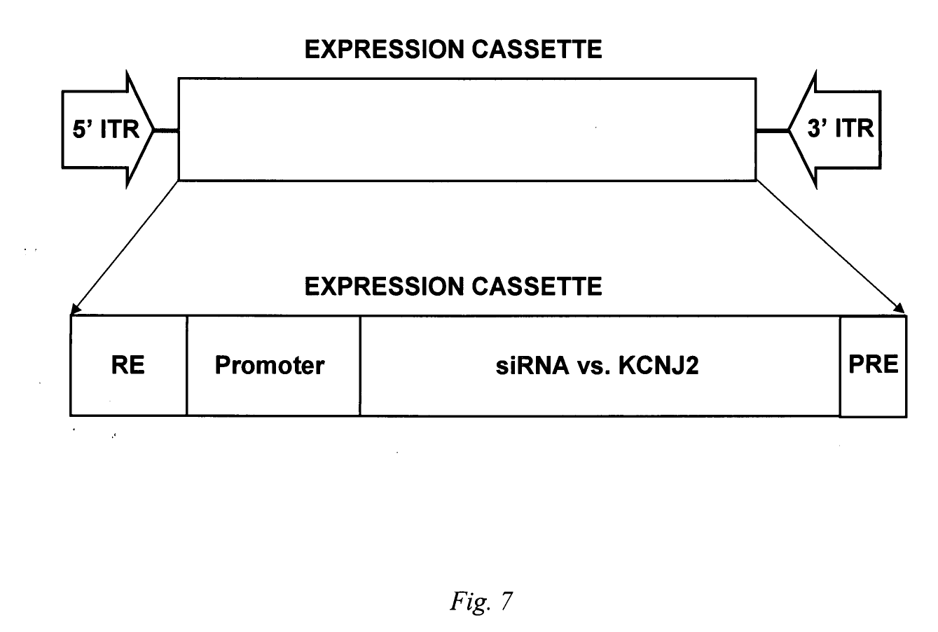

FIG. 7 illustrates a representative viral expression cassette comprising an oligonucleotide that can serve to suppress KCNJ2 according to the present invention. Referring to the figure, ITR denotes inverted terminal repeats; RE is a regulatory element such as drug-sensitive elements; the promoter can be U6 (rodent) or H1 (human) RNA pol III promoter; siRNA vs. KCNJ2 denotes the oligonucleotide siRNA for KCNJ2 (DNA encoding for a short, hairpin RNA transcript that endogenous, intracellular dicer enzyme processes into siRNA); and PRE denotes postregulatory elements.

Suitable vectors, according to the present invention, can be either viral or non viral. Suitable viral vectors include, but are not limited to, adenoviruses, adeno-associated viruses, retroviruses, lentiviruses, herpes simplex viruses, semliki forest virus, vaccinia viruses, and combinations thereof. Other suitable delivery devices include, but are not limited to, polymers, liposomes, nanospheres, and combinations thereof. Furthermore, electroporation, a method for electrically inducing holes in cell membranes, can be used to transfer macromolecules across membranes.

In vitro methods comprise transfection of a host cell with genetic material outside the patient, then transplanting the cells into the patient. Suitable host cells include heart cells (cardiac cells), stem cells and embryonic cells.

For in vivo delivery, methods for targeting non-viral vector genetic constructs to solid tissue or organs, the heart, for example, have been developed such as those described in U.S. Pat. No. 6,376,471.

Vectors suitable for use in connection with the methods of the present invention include viral vectors having a strong eukaryotic promoter operably linked to the polynucleotide, e.g., a cytomeglovirus (CMV) promoter. Suitable viral vectors include, but are not limited to, adenoviruses, adeno-associated viruses, retroviruses, lentiviruses, herpes simplex viruses, vaccinia viruses, Epstein-Barr viruses, coxsackie viruses, sendai viruses, and combinations thereof.

Adenoviruses are linear, double-stranded DNA viruses with a genome of 36 kb. Viral DNA is encapsulated within a protein coat. The adenovirus vectors may be prepared for gene transfer by deletion of the essential E1 gene. This deletion results in a replication-defective virus, because the E1 gene is required for induction of promoters for other early genes. Defective adenovirus is then grown in cell lines that provide E1 protein in trans. Subsequent deletion of a second early gene allows insertion of up to 7.5 kb of foreign DNA in adenovirus vectors. Very recently, gutless adenoviruses have been successfully created and purified. These latest generation AV vectors are characterized by deletion of E1, E2, E3 and E4 (viral coding regions) and may represent promising novel gene therapy vectors. Advantages of gutless adenoviruses over earlier generation AV are that they elicit less immune responses, less toxicity, long-term expression, and a large transgene carrying capacity. Methods on how to generate these vectors have been described in Hardy et al. J of Virology (1997) 1842-1849 and in Sakhuja et al. Human Gene Therapy (2003) 14:243-254.

Adeno-associated viruses (AAV) are small parvoviruses with a 4.7 kb single-stranded DNA genome surrounded by a protein coat. At least 85 percent of adults are immunopositive for MV, but no specific pathology has been linked to this virus. For embodiments utilizing MV, a helper virus such as adenovirus or herpesvirus may be used to produce an infection. MV shows promise because it infects a very wide variety of cells, including both dividing and non-dividing cells. A benefit of MV is that it tends to produce sustained gene expression in transduced cells. MV proteins are not toxic to cells and do not trigger a strong host immune response. For gene therapy, MV vectors are stripped of most wild-type genes. These vectors retain only two 145 bp inverted terminal repeats that are required for viral packaging and integration.

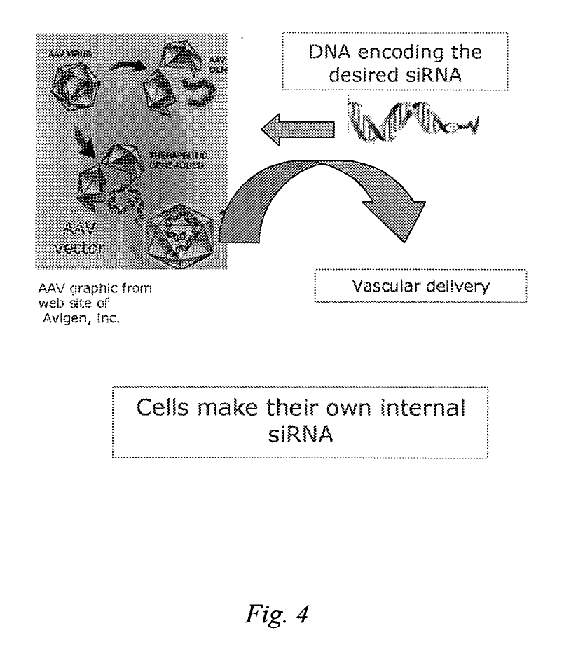

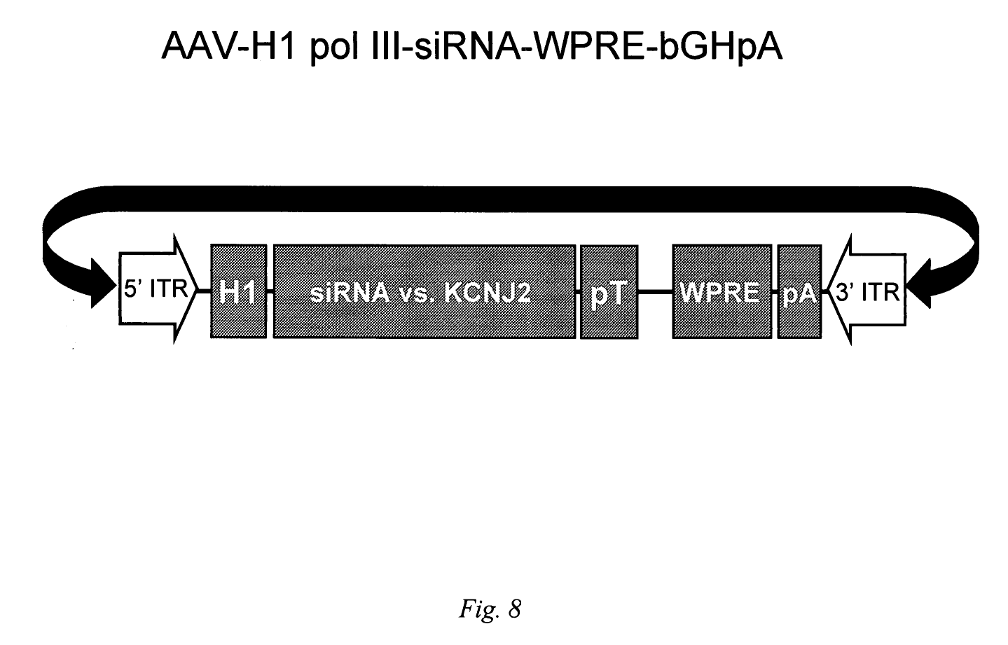

An example of the methods of the subject invention utilizing an adeno-associated viral (MV) vectors is illustrated in FIG. 4. An exemplary MV is a ½ chimeric vector (e.g., XM 086565, MV-CAG-HCN3-WPRE-polyA; control vector MV-CAG-eGFP-polyA). Other exemplary MV vectors are know in the art. See Hauck et al., Molec. Therapy (2003) 7(3):419-425 and U.S. Pat. No. 6,162,796. FIG. 8 is a more specific example utilizing an adeno-associated virus. Referring to FIG. 8, ITR denotes inverted terminal repeats; H1 is a human RNA polymerase III promoter; siRNA vs. KCNJ2 denotes the siRNA against KCNJ2 gene (DNA encoding for a short, hairpin RNA transcript that endogenous, intracellular dicer enzyme processes into siRNA); pT denotes RNA polymerase III transcription termination signal; WPRE denotes a woodchuck post-regulatory enhancer; and pA denotes a poly-adenylation signal.

Retroviral vectors, according to the present invention, include moloney murine leukemia viruses (MMLV). Retrovirus vectors encode three structural proteins (Gag, Pol, and Env) for viral replication, receptor binding, reverse transcription, and viral encapsulation. Retrovirus vectors typically contain two viral long terminal repeats (LTRs) flanking the therapeutic gene. The LTRs include promoter/enhancer functions and sequences required for integration. Vectors also must contain the psi packaging signal. These replication-defective vectors utilize the endogenous retrovirus promoter or viral promoters from cytomegalovirus to drive expression of therapeutic genes.

Lentiviruses are a subclass of retroviruses that express a preintegration complex that controls the infected cell's nuclear import functions. Lentiviruses, such as human immunodeficiency virus type 1 (HIV-1), can replicate in either non-dividing or dividing cells. Lentiviruses infect a variety of cells, including lymphocytes and brain, muscle, retinal, cochlear, heart, liver, and hematopoietic stem cells. Recently, long-term gene transfer into hematopoietic stem cells has been achieved with lentivirus vectors. Lentivirus vectors show promise in correcting inherited genetic disorders in blood cells, since these cells are derived from stem cells.

Herpes simplex virus (HSV) types 1 and 2 are linear double-stranded DNA viruses with genomes of approximately 150 kb and an outer lipid membrane. HSV infects a wide variety of cells and may induce lifelong latent infections by remaining as an episome within cells. The large genome size of HSV permits the insertion of therapeutic DNA sequences up to 30 kb long. Therefore, several therapeutic genes may be placed in tandem in HSV vectors.

Vaccinia viruses are double-stranded DNA viruses with a genome of approximately 200 kb and an outer lipid membrane. Vaccinia virus remains in the cytoplasm of infected cells and uses viral polymerases for both replication and transcription. Vaccinia is a promising vector because the virus infects nearly all mammalian cell types. Vaccinia vectors can accept DNA sequences up to 25 kb long, but they induce a strong cytotoxic T-cell response in tissue.

The oligonucleotide of the subject invention may be operably linked to a promoter. A promoter may be activated by electrical, pharmacological, or other means. Promoters (electrically-responsive, drug-responsive) and other regulatory elements may also be used.

Exemplary promoters include, but are not limited to, alpha-MHC N terminus (2366 bp). Promoters useful for the present invention include both inducible and supressible promoters. Inducible promoters include, but are not limited to, ecdysone-inducible promoter. See U.S. Pat. Nos. 6,214,620 and 6,506,379. Suppressible promoters include, but are not limited to, tetracycline-responsive elements (suppressor). Suitable electrically-responsive promoters are disclosed in United States Patent Application Publication No. U.S. 2003/0204206 A1.

Yet another example of a suitable promoter is the cytomegalovirus (CMV) promoter. The Rous sarcoma virus (RSV) (Davis, et al., Hum Gene Ther 4:151 (1993)) and MMT promoters may also be used. This cassette may be inserted into a vector, e.g., a plasmid vector such as pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The construct or plasmid may also include a selectable marker such as the .beta.-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

Cardiac tissue specific promoters (which allow cardiac myocyte specific expression of transgene of interest, including cardiac stem cells, etc) useful in the practice of the present invention include, but are not limited to, cardiac ankyrin repeat protein (see e.g., U.S. Pat. No. 6,451,594 by Chien et al), alpha-myosin heavy chain gene, beta-myosin heavy chain gene, myosin light chain 2 v gene; United States Patent Application Publication No. U.S. 2002/025577A1 shows a myosin light chain enhancer, followed by either a myosin-heavy chain promoter or a viral promoter and a polynucleotide sequence, myosin light chain 2a gene, cardiac alpha-actin gene, cardiac M2 muscarinic acetylcholine gene, ANF (ANP) Atrial natriuretic factor (or peptide), cardiac troponin C, cardiac troponin 1, cardiac troponin T, and cardiac sarcoplasmic reticulum Ca-ATPase gene.

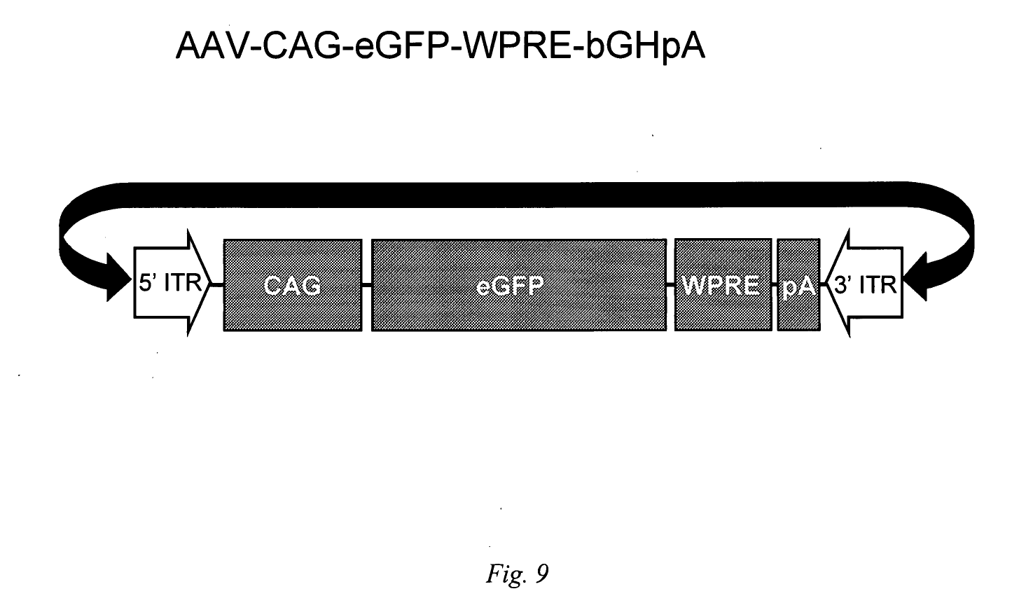

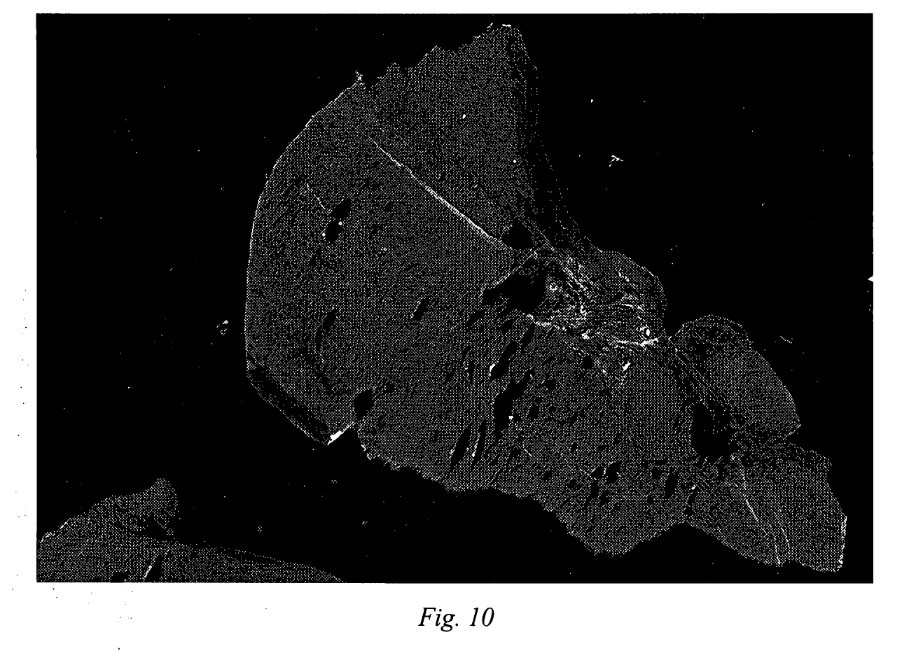

FIG. 9 depicts an AW vector useful for transfecting cells and providing long-term gene expression. Here, a eGFP viral control vector is used in canine gene expression experiments. In FIG. 9, ITR denotes inverted terminal repeats; CAG denotes chicken B-actin promoter/CMV enhancer; eGFP denotes the gene encoding enhanced green fluorescent protein; WPRE is a woodchuck post-regulatory enhancer; and pA denotes pA “poly-adenylation signal”. Use of such a viral vector provides for chronic gene expression as seen in FIG. 10 depicting a fluorescence microscope image demonstrating positive GFP expression 4 weeks after injection of an AAV vector encoding eGFP into canine myocardium (width of image=18 mm).

Arrhythmia and other heart disease may be treated by administering the oligonucleotides disclosed herein in combination with traditional device-based therapies (e.g., pacemakers and defibrillators) and/or together with other gene therapies. As noted above, delivery of the oligonucleotides can also be achieved by non-viral vectors such as polymers, liposomes, and nanospheres, or by physical means such as electroporation. In addition, genetic material can be injected directly into the myocardium as described by Guzman et al., Circ. Res. (1993) 73:1202-1207.

The present invention provides for methods of introducing siRNA at specific locations within the heart using a fluid delivery catheter or lead. Examples of such methods are described in commonly-assigned copending U.S. patent application Ser. Nos. 10/262,046, filed Oct. 2, 2002.

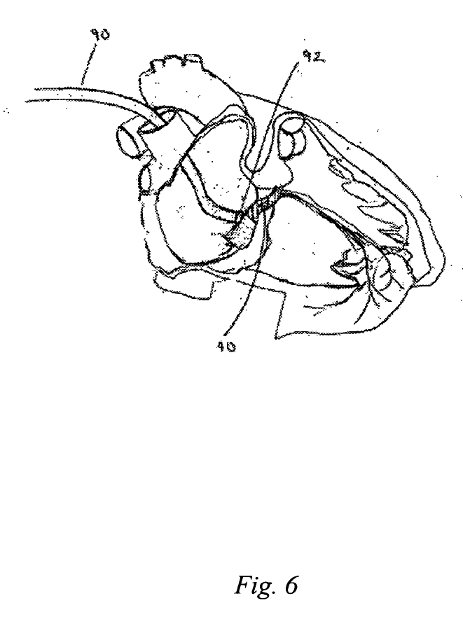

Genetic material may also be delivered by the fluid delivery catheter described in commonly-assigned copending U.S. application Ser. No. 10/423,116, filed Apr. 23, 2003. As an example, FIG. 6 is a schematic diagram of the right side of a heart; similar to that shown in FIG. 1, wherein a guide catheter 90 is positioned for delivery of the genetic construct of the invention. A venous access site (not shown) for catheter 90 may be in a cephalic or subclavian vein and means used for venous access are well known in the art, including the Seldinger technique performed with a standard percutaneous introducer kit. Guide catheter 90 includes a lumen (not shown) extending from a proximal end (not shown) to a distal end 92 that slideably receives delivery system. Guide catheter 90 may have an outer diameter between approximately 0.115 inches and 0.170 inches and is of a construction well known in the art. Distal end 92 of guide catheter 90 may include an electrode (not shown) for mapping electrical activity in order to direct distal end 92 to an implant site near bundle of His 40. Alternatively a separate mapping catheter may be used within lumen of guide catheter 90 to direct distal end 92 to an implant site near bundle of His 40, a method well known in the art.

The present invention may also be used in conjunction with a traditional pacemaker and/or defibrillator. In such embodiments, an implantable device can sense arrthymia via electrical sensing leads, and also administer genetic therapy via a fluid delivery catheter lead. As genetic therapy is not instantaneous, a traditional implantable medical device, like a pacemaker, could be implanted as a safeguard.

A suitable pacemaker useful in connection with the methods described herein comprises a permanently placed pacing lead allowing for gene delivery, but also pacing therapy and monitoring. Such a device can operate to facilitate a particular series of events, namely: bradycardia sensed→pacing→electrically responsive promoter induces formation of silencing siRNA→automaticity→pacing stopped (example of a true hybrid device). Use of an electrically responsive promoter is particularly attractive in conjunction with an electrical pacing lead that also provides for delivery of a fluid to the cardiac tissue, through which a vector for transducing cells with a gene under the control of the electrically responsive promoter can be delivered.

The transplantation of recombinant host cells into the myocardium may also be administered to a subject by well-known surgical techniques for grafting tissue and/or isolated cells into a heart. In general, there are two methods for introducing the recombinant cells into the subject's heart tissue:1) surgical, direct injection; or 2) percutaneous techniques as described in U.S. Pat. No. 6,059,726 (Lee and Lesh, “Method for locating the AV junction of the heart and injecting active substances therein”).

Suppression of KCNJ2 in recombinant host cells prior to introduction into a heart can be detected by such techniques as western blotting, utilizing antibodies specific for inward rectifier potassium channel Kir2.1. Other methods for confirming the suppression of KCNJ2 in transformed cells may involve RT-PCR utilizing primers specific for inward rectifier potassium channel Kir2.1 mRNA or immunofluorescence techniques on transformed cells in culture.

The recombinant host cells can be implanted into any area of the heart where conduction disturbances have occurred. The amount of recombinant cells to be transplanted is determined by the type of heart disease, the overall damage of myocardial tissue and the level of KCNJ2 suppression in the cells to be transplanted. Of particular interest with respect to the cardiac stimulation aspects of the invention, the cells are delivered into a region of heart tissue to be stimulated.

The recombinant host cells may be transplanted by percutaneous methods. If the site of the damaged heart tissue can be accurately determined in a subject by non-invasive diagnostic techniques, the recombinant cells can be injected directly into the damaged myocardial tissue using general methods for percutaneous injections into cardiac muscle well known in the art, and further with respect to the novel, beneficial delivery embodiments provided herein. The amount of recombinant cells necessary to be therapeutically effective will vary with the type of disorder being treated as well as the extent of heart damage that has occurred.

Immunosuppressants may be used in conjunction of transplantation of the recombinant cells not derived from the host to minimize the possibility of graft rejection, e.g., allogeneic or xenogeneic cells.

The methods of the subject invention may also be utilized in combination with other cardiac therapies when appropriate including biological agents including fusion proteins and chemical conjugates and other drugs. Biological agents used to treat certain types of conduction defects can be administered in combination with the oligonucleotides, simultaneously or at different times during the therapy. Biological agents that are suitable for use in a combination therapy include, but are not limited to, growth factors, polynucleotides encoding growth factors, angiogenic agents, calcium channel blockers, antihypertensive agents, antimitotic agents, inotropic agents, antiatherogenic agents, anti-coagulants, beta-blockers, anti-arrhythmic agents, antiinflammatory agents, vasodilators, thrombolytic agents, cardiac glycosides, antibiotics, antiviral agents, antifungal agents, agents that inhibit protozoans, antiarrhythmic agents (used for treatment of ventricular tachycardia), nitrates, angiotensin converting enzyme (ACE) inhibitors; brain natriuretic peptide (BNP); antineoplastic agents, steroids, and the like.

The effects of therapy can be monitored in a variety of ways. Generally for heart block disorders, an electrocardiogram (ECG) or holter monitor is utilized to determine the efficacy of treatment. The contraction of the heart occurs due to electrical impulses that are generated within the heart; an ECG is a measure of the heart rhythms and electrical impulses. Thus ECG is a very effective and non-invasive way to determine if therapy has improved or maintained, prevented, or slowed degradation of the electrical conduction in a subject's heart. The use of a holter monitor, a portable ECG that can be worn for long periods of time to monitor heart abnormalities, arrhythmia disorders, and the like, is also a reliable method to assess the effectiveness of therapy.

Electrophysiology tests involving percutaneous placement of catheters within the heart can also be used to assess the conduction properties of the heart.

Where the condition to be treated with bioactive agent delivery is congestive heart failure, an echocardiogram or nuclear study can be used to determine improvement in ventricular function. Comparison of echocardiograms prior to and after the grafting of recombinant cells into myocardial tissue allows for reliable assessment of treatment. All patents and publications referenced herein are hereby incorporated by reference.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

1. An isolated nucleic acid molecule comprising a nucleic acid as shown in SEQ ID NO: 1.

2. An isolated nucleic acid molecule comprising a nucleic acid as shown in SEQ ID NO: 2.

3. An isolated nucleic acid molecule comprising a nucleic acid as shown in SEQ ID NO: 3

4. An isolated nucleic acid molecule comprising a nucleic acid as shown in SEQ ID NO: 4.

5. An isolated nucleic acid molecule comprising a nucleic acid as shown in SEQ ID NO: 5.

6. An isolated nucleic acid molecule comprising a nucleic acid as shown in SEQ ID NO: 6

7. An isolated nucleic acid molecule comprising a nucleic acid as shown in SEQ ID NO: 7.

8. An isolated nucleic acid molecule comprising a nucleic acid as shown in SEQ ID NO: 8.

9. An isolated nucleic acid molecule comprising a nucleic acid as shown in SEQ ID NO: 9.

10. An isolated nucleic acid molecule comprising a nucleic acid as shown in SEQ ID NO: 10.

11. An isolated nucleic acid molecule comprising a nucleic acid as shown in SEQ ID NO: 11.

12. An isolated nucleic acid molecule comprising a nucleic acid as shown in SEQ ID NO: 12.

13. An isolated nucleic acid molecule comprising a complement of the nucleic acid molecule of any one of claims 1, 2, 3, 4, 5, 6, 7, 8, 9, 10, 11 or 12.

14. A vector comprising the nucleic acid of any one of claims 1, 2, 3, 4, 5, 6, 7, 8, 9, 10, 11 or 12.

15. A host cell genetically engineered to contain the nucleic acid of any one of claims 1, 2, 3, 4, 5, 6, 7, 8, 9, 10, 11 or 12.

16. A method of treating a cardiac disorder comprising administering to a patient in need thereof a therapeutic amount of small interfering RNA that suppresses the expression of the inward potassium rectifier channel 2.1.

17. A method of treating a cardiac disorder comprising administering to a patient in need thereof a therapeutic amount of a vector comprising small interfering RNA wherein said RNA suppress the expression of the inward potassium rectifier channel 2.1.

18. A method of treating a cardiac disorder comprising administering to a patient in need thereof a therapeutic amount of a host cell comprising small interfering RNA wherein said RNA suppress the expression of the inward potassium rectifier channel 2.1.

19. The methods of claim 16, 17, or 18 wherein the small interfering RNA is selected from the group of SEQ ID NOS 1-6.

20. A kit comprising an oligonucleotide having a sequence selected from the group consisting of SEQ ID NOS: 1-6 and a vector for transfecting the oligonucleotide into a host cell.

21. The kit of claim 20, wherein the vector is a viral vector.

22. The kit of claim 21, wherein the viral vector is an MV.

23. The kit of claim 20, further comprising a fluid delivery catheter device.

24. The kit of claim 20, further comprising an implantable pacemaker.

25. The kit of claim 20, wherein the kit is useful in the treatment of arrhythmia.

26. An oligonucleotide comprising a sequence selected from the group consisting of SEQ ID NOS: 1-12.

27. A pharmaceutical composition comprising an oligonucleotide selected from the group consisting of SEQ ID NOS: 1-12 and a pharmaceutically acceptable carrier.

28. A vector useful for transfection or transformation of a host cell comprising the oligonucleotide of claim 26.

29. A host cell comprising the vector of claim 28.

30. A method of treating a cardiac disorder comprising administering to a mammal a therapeutic amount of an oligonucleotide of claim 26.

31. The method of claims 16, 17, and 18 wherein the cardiac disorder is arrhythmia.

32. The method of claims 16, 17, and 18 wherein a fluid delivery catheter is used to administer the oligonucleotide to the mammal.

33. A method of correcting arrhythmia in a mammal by administering a therapeutic amount of siRNA wherein said siRNA suppresses the expression of the KCNJ2 gene.


SEQUENCE LISTING (12 sequences)

SEQ ID NO: 1 - DNA, 21 bp, Artificial
  siRNA target sequence within the mRNA sequence for Kir2.1
aacggtacctcgcagacatct

SEQ ID NO: 2 - DNA, 21 bp, Artificial
  siRNA target sequence with the mRNA sequence for Kir2.1
aacttacgaagtccccaacac

SEQ ID NO: 3 - DNA, 21 bp, Artificial
  siRNA target sequence with the mRNA sequence for Kir2.1
aaccataggctatggtttcag

SEQ ID NO: 4 - DNA, 21 bp, Artificial
  siRNA target sequence with the mRNA sequence for Kir2.1
aatgaagttgccctcacaagc

SEQ ID NO: 5 - DNA, 21 bp, Artificial
  siRNA target sequence with the mRNA sequence for Kir2.1
aatgttgggtttgacagtgga

SEQ ID NO: 6 - DNA, 21 bp, Artificial
  siRNA target sequence with the mRNA sequence for Kir2.1
aatgccgtgattgccatgaga

SEQ ID NO: 7 - DNA, 57 bp, Artificial
  DNA sequence encoding for RNA hairpin transcripts corresponding to each of the siRNA candidates targeting Kir2.1
aacggtacctcgcagacatctttcaagagaagatgtctgcgaggtaccgtttttttt

SEQ ID NO: 8 - DNA, 57 bp, Artificial
  DNA sequence encoding for RNA hairpin transcripts corresponding to each of the siRNA candidates targeting Kir2.1
aacttacgaagtccccaacacttcaagagagtgttggggacttcgtaagtttttttt

SEQ ID NO: 9 - DNA, 57 bp, Artificial
  DNA sequence encoding for RNA hairpin transcripts corresponding to each of the siRNA candidates targeting Kir2.1
aaccataggctatggtttcagttcaagagactgaaaccatagcctatggtttttttt

SEQ ID NO: 10 - DNA, 57 bp, Artificial
  DNA sequence encoding for RNA hairpin transcripts corresponding to each of the siRNA candidates targeting Kir2.1
aatgaagttgccctcacaagcttcaagagagcttgtgagggcaacttcatttttttt

SEQ ID NO: 11 - DNA, 57 bp, Artificial
  DNA sequence encoding for RNA hairpin transcripts corresponding to each of the siRNA candidates targeting Kir2.1
aatgttgggtttgacagtggattcaagagatccactgtcaaacccaacatttttttt

SEQ ID NO: 12 - DNA, 57 bp, Artificial
  DNA sequence encoding for RNA hairpin transcripts corresponding to each of the siRNA candidates targeting Kir2.1
aatgccgtgattgccatgagattcaagagatctcatggcaatcacggcatttttttt